(19) United States
(12) Patent Application Publication
(10) Pub. No.: US 2017/0340291 A1
(43) Pub. Date: Nov. 30, 2017

(54) NON-INVASIVE MEDICAL ANALYSIS BASED ON TS FUZZY CONTROL

(71) Applicant: Mohamed Issam AYARI, Tunis (TU)

(72) Inventors: Saif ALHAISOUNI, Saudi Arabia (SA); Adnan ALAHMADI, Saudi Arabia (SA); Ayoub ALZUMAYA, Saudi Arabia (SA)

(21) Appl. No.: 15/537,850

(22) PCT Filed: Jan. 7, 2015
(86) PCT No.: PCT/IB2015/050124

§ 371 Date: Jun. 19, 2017

(51) Int. Cl. A61B 5/00; A61B 5/1455; A61B 5/145

(52) U.S. Cl. CPC A61B 5/7264; A61B 5/7282; A61B 5/4872; A61B 5/14532; A61B 5/4362; A61B 5/1455; A61B 5/4845

(57) ABSTRACT

Measuring and monitoring the glucose and other blood elements through high accuracy non-invasive method. The proposed approach is based on TS Fuzzy model in order to modeling the proportion of each component in blood. This model determine the degree of absorption for each component and allows the measurement of glucose with more degree of accuracy.

The proposed method use Monte Carlo Simulation in order to measuring the path-length of photon in different tissues. We develop a modified Monte Carlo Algorithm based on TS Fuzzy model. Then, we can determine several concentration of elements in blood by using this method. On the other hand, we propose a novel laser diode in order to measuring concentrations of different blood components. The proposed diode give a de-multiplexing of enter light to several wavelengths needed for determination of element concentration in blood by using Bear Lambert Law. Two methods are proposed or this end, Silicon On Isolator method and Photonic Crystal method. We develop Multiplexer/De-Multiplexer based on one of these method in order to decompose the incident light to wavelength needed by system. Third part, concern the modification of regression approach by using TS Fuzzy model in order to give more accuracy in measurement of glucose level in blood. The regression methods studies are linear regression and ordinary least square regression.

### INVENTION'S BACKGROUND

Bioelectronics' Innovation.

The non-invasive glucose meter based on absorption still complain of a lack of precision necessary to become certified and safety in order to replace the strip devices. This innovation offers solutions to these problems. In fact, we design a new diode: Photonic Crystal Diode, which enables us to disperse light into multiple wavelengths. Consequently, we can dispense of several diodes in order to measure the concentrations of all elements needed to make a medical analysis for diabetic.

In addition, we can use multiple wavelengths in order to obtain high accuracy in measurement of the concentration of glucose in the blood.

We propose a new mathematical model of the optical absorption based on fuzzy logic, which enables us to measure the proportion of each chemical element in the blood concentration with high accuracy, depending on its vulnerability by the absorbed wavelength.

### MAIN DESCRIPTION

Using the absorption property of chemical elements in blood in order to doing medical analysis through thumbprint, depending on the Bear-Lambert law in order to calculate the concentration of these elements in the blood. In fact, we design a new photonic crystal diode by Photonic Crystal technology—or silicon-on-insulator technology (Silicon On Insulator) which enables us to disperse light by wavelength. The advantage of this technology is to measure the concentration of several chemical elements by absorption at the same time. In addition, we propose a new mathematical model of absorption based on fuzzy logic control

This model enables us to measure the glucose level in blood with high accuracy by using a range of wavelengths, which are affected by glucose. The advantage of this model is reduction of error rate and measurement of concentration for a range of chemical elements in the blood at the same time.

For more accuracy in glucose level measurement, the photon path-length in tissues is determined through Monte-Carlo Simulation. In all steps we use the proposed Fuzzy logic model of absorption. In addition we can use the proposed model in determination of linear or other regression functions used in computing of glucose level in blood.

Accordingly, we can precise the appropriate treatment based on results obtained automatically by this devise through print-electronic doctor-detection of epidemic diseases and glucose level and fat level in blood.

### BRIEF FIGURES EXPLANATION

FIG. 1 shows the principal of proposed diode based on photonic crystal technic or Silicon On Insulator one, we observe how we can disperse the light in a set of wavelength allowing to chemical element in blood to absorb the affected wavelength.

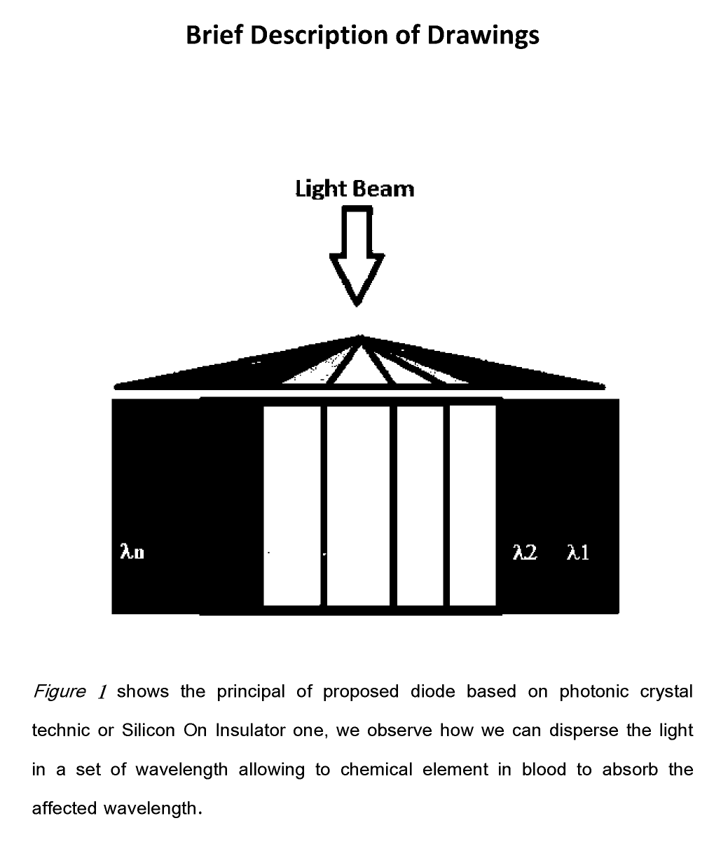

FIG. 2 illustrates the proposed system measurement based on new photonic crystal diode and the proposed fuzzy logic model of absorption allowing the control of each element in blood in order to do medial analysis for diabetics and other disease.

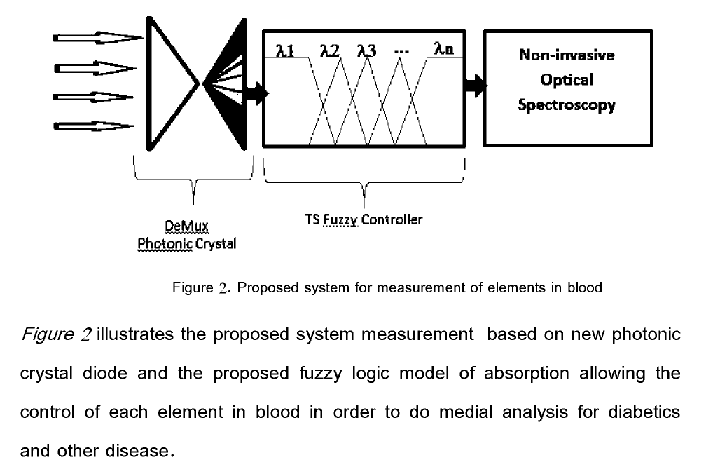

### DETAILED DESCRIPTION

Using the absorption law in order to do the medial analysis, according to Bear Lambert law for measurement of concentration of chemical element in blood. In fact, we propose this innovation of new photonic crystal diode which allow to disperse the incident light in a set of wavelengths. Consequently, each element to measure absorb the correspondent wavelength and we can by this proceed to determine its concentration in the blood. Mathematically, we propose the absorption model based on TS Fuzzy Logic in order to determine the proportion of each element in blood with high accuracy. In addition, we can use this model or measuring the glucose level through a set of wavelength.

For more accuracy in glucose level measurement, the photon path-length in tissues is determined through Monte-Carlo Simulation. In all steps we use the proposed Fuzzy logic model of absorption. In addition we can use the proposed model in determination of linear or other regression functions used in computing of glucose level in blood.

1- Non-invasive device for determine and control of glucose level and other element in blood by proposition of TS Fuzzy logic model of absorption in order to measure the concentration of chemical element in the blood with more accuracy based on Bear-Lambert Law.

2- design of new diode based on photonic crystal technic or silicon on insulator technic in order to dispersing the light in a set of wavelength which allow to control the absorption for each element in blood.

3- Investigation of a new mathematical model of absorption in order to measure with accuracy the level of each element in blood. The aim of this result is to make the medial analysis for diabetics and other objectives. This model allows to measure the glucose level in the blood by using a range of wavelength which affect the glucose by control of each proportion. The advantage of this method is to give more accuracy in measurement of glucose level or other element in blood by non-invasive technology.
In addition, we can measure several elements in blood in the same time.

4- using Monte-Carlo Simulation for measuring the path-length of photon in tissue. The TS Fuzzy model allow to determine this path-length.

5- Adoption thumbprint to carry out the necessary measurement and identification of all ratios of basic and exotic blood elements through absorption technology.

6- design of electronic doctor system in order to precise the appropriate treatment according to absorption of element based on the proposed devise

7- detect infectious diseases through optical absorption technique

8. detect glucose and fat level in blood through optical absorption technique.

9- Genetic linkage between the couple and determine prevailing qualities that may appear on the children identified with gender compatibility before marriage by optical absorption technique

10- determine the foetus by optical absorption technique

11- detection pregnancy by optical absorption technique

12- discovery of injecting drug by optical absorption technique
